(19) United States
(12) Patent Application Publication
(10) Pub. No.: US 2019/0117100 A1
(43) Pub. Date: Apr. 25, 2019

(54) Wearable Multi-Lead Diagnostic ECG Recording Device

(71) Applicant: Cardiac Markers, LLC, Stillwater, MN (US)

(72) Inventors: Bruce Ernest Rollie, Stillwater, MN (US); Carl Edward Horton, Ridgefield, WA (US); Jon Val Barron, Valley Center, CA (US)

(21) Appl. No.: 16/165,391

(22) Filed: Oct. 19, 2018

Related U.S. Application Data
(60) Provisional application No. 62/574,566, filed on Oct. 19, 2017.

(51) Int. Cl. A61B 5/0404; A61B 5/00; A61B 5/044; A61B 5/0408

(52) U.S. Cl. CPC A61B 5/0404; A61B 5/681; A61B 5/044; A61B 5/0022; A61B 2562/0209; A61B 5/04087; A61B 5/0006; A61B 2562/14; A61B 2562/04; A61B 5/04085

(57) ABSTRACT

A wearable ECG device includes a band having a first and a second ECG electrode, the first and the second ECG electrodes electrically connected as a common electrode. A device body is removably coupled to the band, and has a face and a display on a first side thereof, and a third ECG electrode on an opposite face thereof. A clasp is coupled to the band to secure the device around a wrist of a user, the clasp having a fourth ECG electrode, the fourth ECG electrode insulated from the band.

### CROSS REFERENCE TO RELATED APPLICATION

The present application is based on and claims the benefit of U.S. provisional patent application Ser. No. 62/574,566, filed Oct. 19, 2017, the content of which is hereby incorporated by reference in its entirety.

### BACKGROUND

In electrocardiography, the electrical activity of the heart is recorded. During a heartbeat, an electrical signal starts at the top of the heart in the sinoatrial (SA) node (the pacemaker), then moves down through the heart through a network of conducting cells. Each cell transmits the signal to adjacent cells, thereby coordinating the contraction of the chambers of the heart. This movement of electrical signals through the heart causes electrical changes on the skin that can be detected by electrodes placed on a person's body.

An electrocardiogram (ECG or EKG) is a record of this electrical activity of the heart as detected through resultant electrical changes on the skin, and is read in the form of lines called “tracings.” The tracings may be analyzed to determine the health of the heart. For instance, an ECG may be used to detect irregular heartbeats, infarctions (obstruction of the blood supply), abnormalities in the physical structure of the heart, to determine side effects of medications, or to check the functioning of artificial pacemakers.

A standard ECG monitor uses 12 leads, or lines through the body along which a change in electrical potential is measured. Each lead provides information on a different area of the heart. Ten electrodes are used for a 12-lead ECG, and each of the 12 leads extends between a different electrode pair. The electrodes of a 12-lead monitor are typically disposed in pads attached to specific locations on a person's body by a conducting paste or gel, and are connected to the monitor by wires. Because the electrodes must be placed correctly, and because the user remains connected to the monitor by wires throughout the recording, a 12-lead monitor is typically used in medical settings.

Ongoing ECG monitoring outside of the medical setting is often useful. For instance, some heart conditions are only observable under certain conditions, such as when a person is exercising or under stress. Other conditions occur intermittently, necessitating that a person be monitored over a period of time in order to record the irregularity when it occurs. Ambulatory ECGs record the electrical activity of the heart outside of a medical setting during a person's usual activities. Ambulatory monitors are typically small enough to be carried or attached to the body by a strap, and are connected to a user via wires attached to electrodes placed on the body. Ambulatory monitors are typically 5-lead monitors and provide less information than 12-lead monitors.

Event monitors are useful for recording the electrical activity of a heart at a user's discretion, for instance, when symptoms occur. Event monitors are typically relatively small, and are hand-held. To record an ECG, the user presses electrodes built into the monitor with the fingers of opposite hands, or presses the monitor to his chest. These monitors are convenient and easy to use, as placement of wired electrodes is not required. However, event monitors use only two electrodes, and are thus 1-lead monitors as the drop in electrical potential between one pair of electrodes is measured. This limits the amount of information that can be obtained from this type of monitor. Thus, there is a need for a portable and easy-to-use event monitor that offers more information than typical 1-lead event monitors.

### SUMMARY

In one embodiment, a wearable ECG device includes a band having a first and a second ECG electrode, the first and the second ECG electrodes electrically connected as a common electrode. A device body is removably coupled to the band, and has a face and a display on a first side thereof, and a third ECG electrode on an opposite face thereof. A clasp is coupled to the band to secure the device around a wrist of a user, the clasp having a fourth ECG electrode, the fourth ECG electrode insulated from the band.

In another embodiment, an ECG device includes a device body having a face and a display on a first side thereof, and an ECG electrode on an opposite face thereof, and an electro-mechanical matching patch comprising an electrode configuration for contact with skin of a user, the electro-mechanical matching patch configured to receive the device body therein.

In another embodiment, a method of operating a wearable ECG device includes removably coupling device body having a single electrode to a device receptacle, and providing three electrodes in the device receptacle. Two of the three electrodes are electrically connected as common electrodes, and a third of the three electrodes is insulated from the two of the three electrodes and the single electrode. The two common electrodes are contacted with two parts of a user body, and the single electrode and the third of the three electrodes with other parts of a user body.

### BRIEF DESCRIPTION OF THE DRAWINGS

The disclosed subject matter will be further explained with reference to the attached figures, wherein like structure or system elements are referred to by like reference numerals throughout the several views.

FIG. 1 shows a schematic view of two exemplary electrode placement patterns for 12-lead ECG monitoring of the prior art.

FIG. 2 shows a front view of an exemplary 1-lead ECG monitor of the prior art.

FIG. 3 shows a schematic view of a lead formed by the monitor of FIG. 2.

FIG. 4 shows a schematic view of three leads formed by an exemplary 3-lead ECG monitor of the present disclosure.

FIG. 5 shows a perspective view of an exemplary ECG monitor of the present disclosure in a fastened state.

FIG. 6 shows a top perspective view of the ECG monitor of FIG. 5 in an unfastened state.

FIG. 7 shows a bottom perspective view of the ECG monitor of FIG. 5 in an unfastened state.

FIG. 8 shows a schematic view of the ECG monitor of FIG. 5 fastened about a user's wrist and in communication with local and remote controllers.

FIG. 9 shows a schematic view of the ECG monitor of FIG. 8 in contact with a user's ribcage.

FIG. 10 shows a perspective view of an ECG monitor device according to another embodiment of the present disclosure.

FIG. 11 shows a top view of the device of FIG. 10.

FIG. 12 shows a bottom view of the device of FIG. 10.

FIG. 13 shows a partial view of a front of the device of FIG. 10 showing positioning of digits on electrodes thereof.

FIG. 14A shows a partial view of a user's chest and arm and a coupled patch according to another embodiment of the present disclosure.

FIG. 14B shows a partial view of a user's chest and arm and a coupled patch according to another embodiment of the present disclosure.

FIG. 15 shows top, bottom, and side elevation views of another embodiment of the present disclosure.

FIG. 16. shows the device of FIG. 15 in place on a user's chest.

While the above-identified figures set forth one or more embodiments of the disclosed subject matter, other embodiments are also contemplated, as noted in the disclosure. In all cases, this disclosure presents the disclosed subject matter by way of representation and not limitation. It should be understood that numerous other modifications and embodiments can be devised by those skilled in the art which fall within the scope and spirit of the principles of this disclosure.

The figures may not be drawn to scale. In particular, some features may be enlarged relative to other features for clarity. Moreover, where terms such as above, below, over, under, top, bottom, side, right, left, etc., are used, it is to be understood that they are used only for ease of understanding the description. It is contemplated that structures may be oriented otherwise.

### DETAILED DESCRIPTION

A multi-lead ECG monitor of the present disclosure is a portable and easy-to-use ECG recording device that allows a user to record 1-lead, 2-lead, or 3-lead ECGs without disruption to a user's activities or routine. A 3-lead ECG provides information about the electrical activity of more areas of a user's heart, and is thus more useful, than a 1-lead or 2-lead event monitor for medical diagnosis.

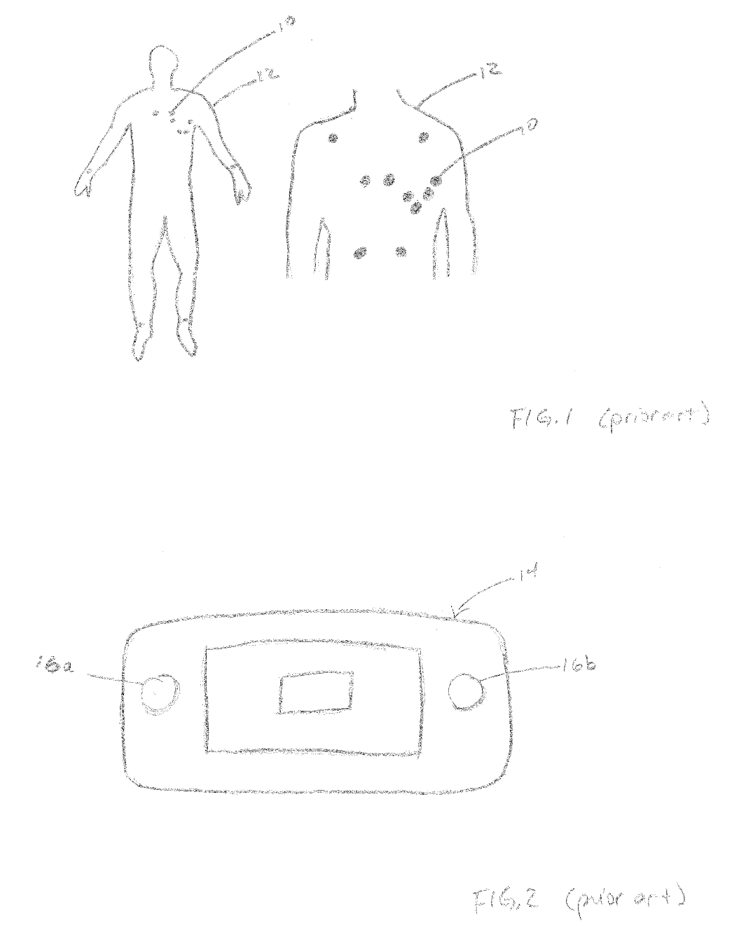

A 12-lead ECG monitor of the prior art requires the application of ten electrodes 10 to various specific locations on a user's body 12, as shown in FIG. 1. Electrodes 10 are typically secured in contact with the skin by pads that are adherable to the skin. The electrodes 10 are connected to a monitor (not shown) somewhat remote to the body by wires (not shown), making user activity cumbersome. Two patterns of electrode 10 placement about the body 12 are shown, but many suitable patterns are known in the art.

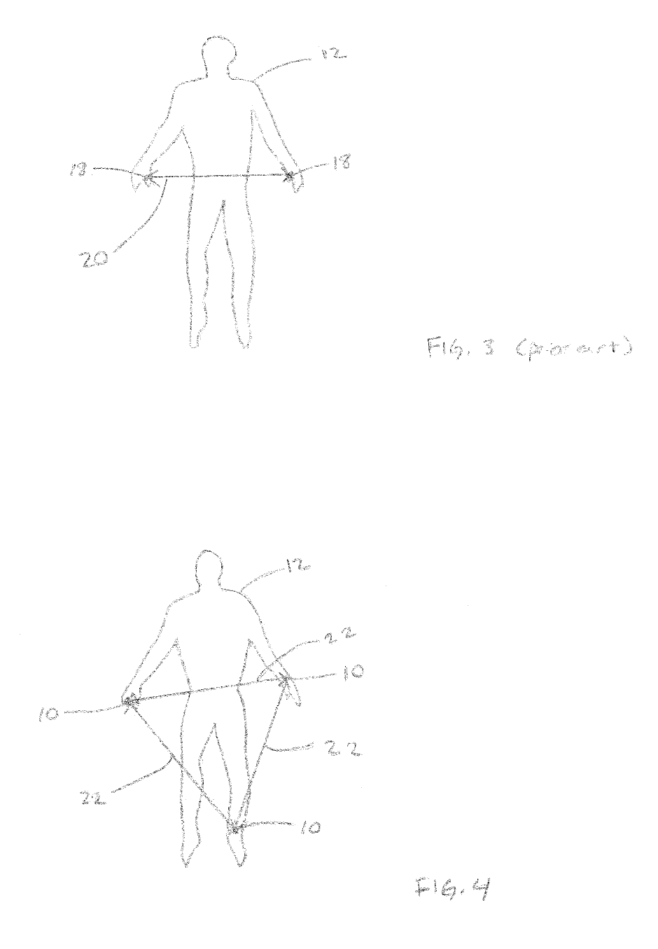

A 1-lead monitor 14 of the prior art is shown in FIG. 2 to have two contact pads 16a, 16b that act as electrodes. Typically, monitor 14 is configured such that a user places each thumb 18 on a different contact pad 16a and 16b. A single lead 20 is thereby formed through body 12 between the two contact pads 16a, 16b, as shown in FIG. 3. The single lead 20 provides information about the electrical activity of a smaller area of the heart than a 12-lead ECG, thus, the diagnostic applications of 1-lead monitor 14 are limited.

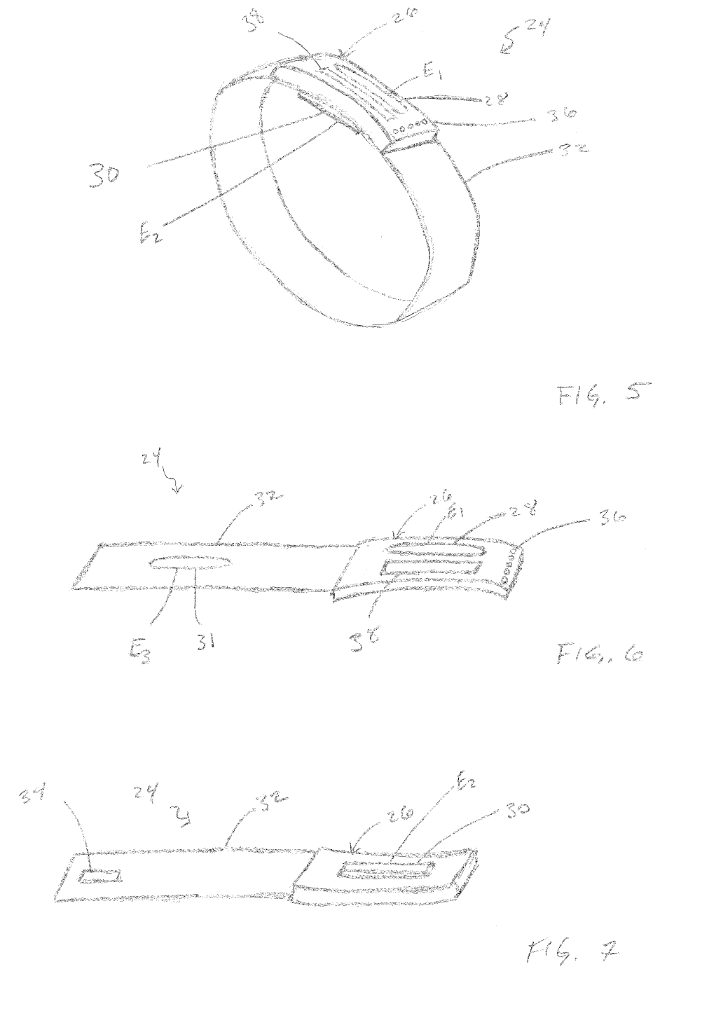

FIG. 4 shows an example pattern of electrode 10 placement for 3-lead monitoring. The use of three electrodes 10 allows a user to measure drops in electrical potential between three sets of electrode pairs, i.e., across three leads 22. Many suitable patterns of electrode 10 placements about the body for 3-lead monitoring are known in the art.

An illustrative 3-lead ECG monitor 24 of the present disclosure is shown in FIGS. 5-7. The monitor 24 includes a unit 26 in which is disposed a recording device (not shown) that is electrically coupled to a finger contact pad 28 (E₁), a wrist contact pad 30 (E₂) (FIG. 7), and another skin contact pad 31 (E₃). Contact pads 28, 30, and 31 may be reusable electrodes. Electrodes may be conductive metal sensors, dry pad sensors, or any electrode suitable for detecting electrical changes on the skin caused by electrical activity of the heart. Unit 26 may have an International Protection (IP) rating of IPX7, and thus be immersible in water to a depth of up to one meter for up to 30 minutes without damage to unit 26.

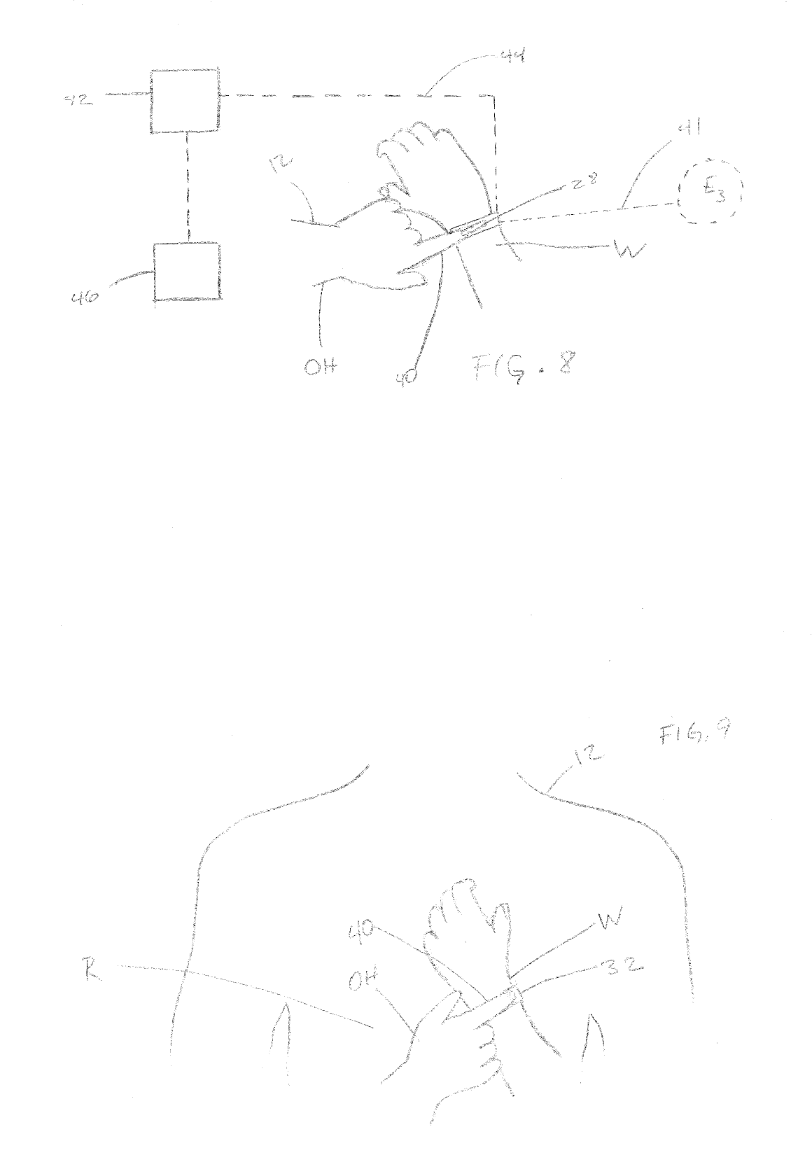

Unit 26 may be attached to either of a user's wrists by a wristband 32 having a closure 34, such that the wrist contact 30 is in contact with the user's wrist. Wristband 32 may be of hypo-allergic material(s) that are ISO 10993 compliant, or of any suitable material. The wristband closure 34 may be a clip, magnet, buckle, etc. Wristband 32 may include a circuit (not shown) that is closed when the wristband 32 is fastened as shown in FIG. 5, and is opened when the wristband 32 is unfastened as in FIGS. 6 and 7. The circuit may be used, for instance, to detect when the monitor 24 is put on or removed. An electrode E₃ may be positioned on wristband 32 as described below.

Monitor 24 may also include an LED array 36 to notify user of test progress, test results, test quality, ECG signal quality, battery life, and the like. LED array 36 may include LEDs of more than one color. Additionally or alternatively, the monitor 24 may include a digital display 38 for the above-mentioned notifications, for notifications such as time, date, and heart rate, and for device functions (e.g., displaying test results or the availability of test results). LED array 36 and digital display 38 may have any suitable shape, and be disposed in or on unit 26 in any suitable configuration. Unit 26 may also include an accelerometer, a gyroscope, and/or other detectors. Unit 26 may also include a haptic feedback motor or emit sounds for user notifications such as those described herein.

In an illustrative embodiment, monitor 24 may be powered by a replaceable and/or rechargeable power supply (not shown), such as a battery or capacitor. Power supply may provide power to monitor 24 for at least about five days, and be recharged in up to about two hours. It is understood that any suitable power supply having any suitable battery life and recharging characteristics may be used.

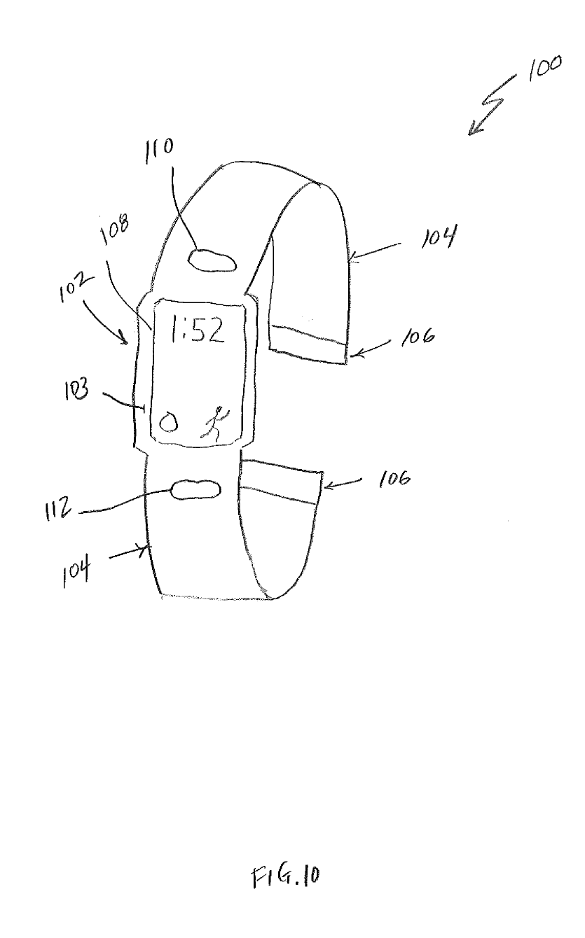

To begin recording of a user's heart activity, the user first places the monitor 24 about one of the user's wrists W. The user then places a finger 40 from the user's other hand OH on finger contact 28 as shown in FIG. 8, thus forming a lead across which a heart's electrical activity may be measured (from finger contact pad 28 (E₁), through the user's body 12, to wrist contact pad 30 (E₂)). In an illustrative embodiment, a third contact pad 31 (E₃) may be positioned on user's torso or leg. Alternatively, third contact pad 32 (E₃) may be positioned on wristband 32. User may place wrist W against user's cheek, ribcage R, or other suitable location on user's body 12 so that contact pad 31 is in contact with user's skin, as shown in FIG. 9. In this way, three leads are formed between the finger contact 28 (E₁), wrist contact 30 (E₂), and the third contact pad 31 (E₃) (see FIG. 4, for example). The third electrode E₃ may be coupled to the recording device via Bluetooth or any suitable wired or wireless system 41. The recording device of monitor 24 may include a data acquisition system (DAS) configured to collect at least about 128-1024 data samples per second from contacts 28, 30, and 31 (E₁, E₂, and E₃). Prior art portable ECG event monitoring systems collect only about 120 data samples per second. The higher sampling rate of monitor 24 is useful in medical and diagnostic applications. The DAS may have a common-mode rejection greater than about 100 db up to frequencies of about 1 KHz, a maximum offset voltage of about 50 uV, and input voltage noise of about 0.05 Hz to about 150 Hz. The DAS may have a signal amplitude of up to about 5 mV with a DC component of up to about +/−300 mV, and a 16 bit signal resolution. Additionally, the DAS may include a 50/60 Hz notched filter with an option to turn the filter off, and may include an option to select a preferred filter design.

Monitor 24 may be configured to transmit ECG information collected by the recording device to a computer, mobile phone, or other device containing a controller 42 such as a microprocessor or the like, either through a wired or a wireless system 44. The device is typically in the user's possession or on the user's person, but may be in any useful location. Controller 42 may receive ECG information from monitor 24, and run algorithmic software that displays or otherwise reports related information, such as ECG traces, analysis, health warnings, and the like. Controller 42 may transmit ECG and/or related information through a wired or wireless system to a remote controller 46 for storage, additional processing, and/or analysis. In certain embodiments, controller 42 may transmit ECG and/or related information to remote controller 46 over electronic mail. Data transmission from monitor 24 to controller 42 and/or data transmission from controller 42 to remote controller 46 may be encrypted.

The 3-lead ECG monitoring and high data-sample speed allowed by the disclosed monitor 24 is suitable for medical and diagnostic purposes. In particular, for instance, monitor 24 is suitable for detecting atrial fibrillation and sudden cardiac arrest.

Although the subject of this disclosure has been described with reference to several embodiments, workers skilled in the art will recognize that changes may be made in form and detail without departing from the spirit and scope of the disclosure. In addition, any feature disclosed with respect to one embodiment may be incorporated in another embodiment, and vice-versa.

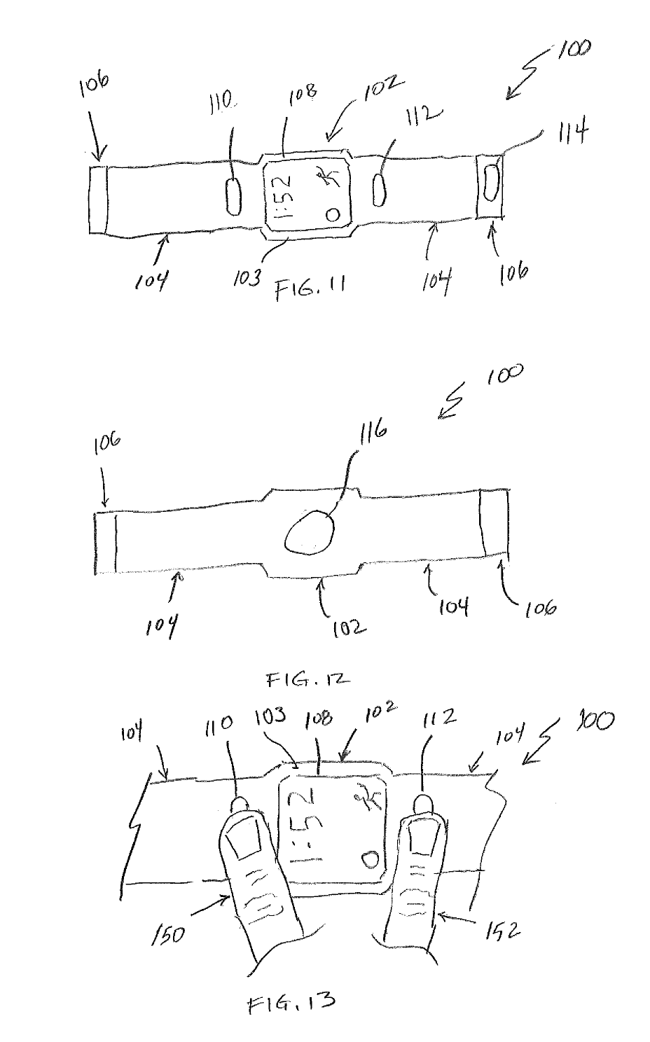

FIG. 10 is a perspective view of another embodiment of a wearable device 100 which includes three-lead direct ECG measurement. Device 100 comprises a device body 102 having a face 103, the body 102 integral with or otherwise removably coupled to a device receptacle such as band 104. The band 104 has a clasp or other fastening device 106 for securing the device 100 to, for example, a wrist of a user. Face 103 may include display screen 108 for display of various and different information, including by way of example only and not by way of limitation, time, activity meter, type of activity, heart rate, ECG information, and the like. It should be understood that the display 104 may display other or different information or in different formats without departing from the scope of the disclosure.

Device 100 differs from device 24 described above in that device 100 comprises four electrodes instead of three electrodes. The four electrodes include, in one embodiment, two electrodes 110 and 112 positioned in one embodiment on either side of face 102 on band 104. The electrodes 110 and 112 are in one embodiment electrically connected to form a common pair of electrodes 110, 112.

FIG. 11 shows the device 100 of FIG. 10 as it appears in a top, unclasped view, showing the outside, or non-wrist facing, side of the device 100. When worn, the common electrodes 110, 112 are not in contact with the wrist of a user. Common electrodes 110, 112 are shown in the band 104. A third electrode 114, electrically separated from common electrodes 110, 112, is shown on the clasp 106 on the outside (non-wrist facing) side of the band 104. Electrode 114 is in one embodiment insulated from the inside (wrist-facing) of the band 104, and is also not in contact with a wrist of a user.

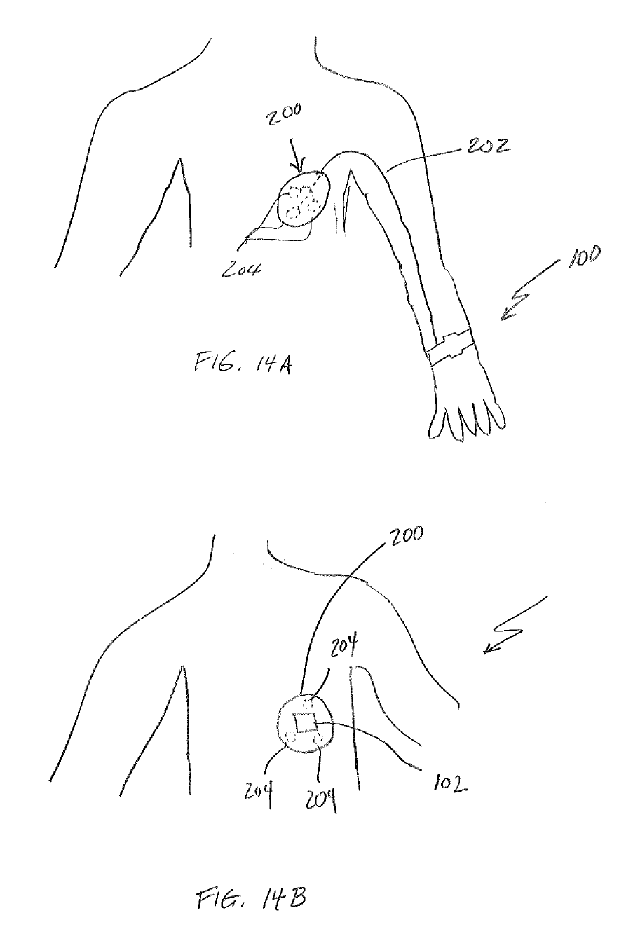

FIG. 12 shows the device 100 of FIG. 10 as it appears in a bottom, unclasped view, showing the inside, or wrist-facing, side of the device 100. Fourth electrode 116 is positioned in one embodiment opposite display 108 of face 102, to contact a wrist of the user when device 100 is worn on the user's wrist. Common electrodes 110, 112 and third electrode 114 are not visible as they are on the outside (non-wrist facing) side of the device 100. Although fourth electrode 116 is shown opposite display 108 of device 100, it should be understood that the placement of fourth electrode 116 may be different provided that fourth electrode 116 is positioned to contact a wrist of the user when the device 100 is being worn.

The four electrodes combine to allow a three-lead direct ECG to be obtained from the wrist and other contact points similar to the three-lead ECG with three electrodes described above. However, the two common electrodes 110, 112 on the non-wrist facing side of the device 100 provide a better ECG for at least several reasons. First, the use of two digits to connect two physically separate but electrically common electrodes allows a more stable platform for contact than a single digit. Two digits are much easier to stabilize than a single digit. The use of two contact points improves stability of the electrode contact with the user's opposite hand, which in turn reduces muscle artifact and improves the ECG signal. The two contact electrodes reduce arm and/or hand muscle movements which can lead to artifacts from muscle tremors or the like.

FIG. 13 shows a partial view of the device 100 in use to obtain an ECG reading. To obtain an ECG reading using the device 100, the third electrode is positioned at a contact point on the user's body, such as but not limited to the chest or leg (see FIG. 9 for example only). Then, the user places two digits 150, 152 of the user's opposite hand on the common electrodes 110, 112, respectively. In this configuration, all four electrodes 110, 112, 114, and 116 maintain contact with the user's skin, and provide for the three-lead ECG to be obtained. In one embodiment, the user places the index finger and thumb of the opposite hand on the electrodes 110, 112. Using the thumb and index finger improves tracing and stabilizes as discussed above. While a thumb 150 and index finger 152 are shown in FIG. 13, it should be understood that any two digits of the user's opposite hand on the common electrodes 110, 112 will result in a more stable contact and therefore better ECG reading.

The use of two common electrodes 110, 112 and their positioning on a non-wrist facing side of the wearable device 100 further allows the wearable device 100 to be wearable on either wrist of a user without any reconfiguration. It should further be understood that the shape of the electrodes 110, 112, 114, and 116 is representative, but that the shape and size of the electrodes may be changed without departing from the scope of the disclosure.

Each of the electrodes 110, 112, 114, and 116 is electrically coupled to an ECG recording device (not shown). Electrodes 110, 112, 114, and 116 may be conductive metal sensors, dry pad sensors, or any electrode suitable for detecting electrical changes on the skin caused by electrical activity of the heart. Recording device and wearable device 100 may have an International Protection (IP) rating of IPX7, and thus be immersible in water to a depth of up to one meter for up to 30 minutes without damage thereto.

In one embodiment, the electrodes may be formed from graphene instead of traditional steel or alloys. It should be understood also that placement of the common electrodes may be changed without departing from the scope of the disclosure. For example, the common electrodes 110, 112 could be positioned on the face 102, or on a same side of the band 104 as each other. It is sufficient that the common electrodes 110, 112 are close enough together to allow for a stable positioning of, for example, two digits of a user's hand opposite the wrist on which the device 100 is worn. While a wrist device 100 is shown and described, it should be understood also that the device need not be worn on the wrist, although for common ECG body contact points, a wrist wearable device provides ease of contact points.

Once an ECG is obtained, embodiments of the present disclosure provide for numerous possibilities for its use. A user is not typically competent to interpret his or her own ECG. Accordingly, in one embodiment, the obtained ECG reading is communicated to a separate diagnostic location. Such communication may be via wireless communication (e.g., over a cellular telephone or wireless network using integrated cellular or wireless technology), through a physical connection of the wearable device 100 to a computer for downloading ECG reading(s) from the device to a computer that can then provide the reading(s) to the diagnostic location. Alternatively, wearable device 100 may be paired with another portable electronic device, such as a cellular telephone or other mobile device.

There are different types of ECG monitoring and recording, for example, (1) user-initiated event recording, and (2) looping or continuous monitoring (also known as Holter monitoring). A Holter Monitor device typically records non-stop for 24-48 hours. After recording is done the data is downloaded from the monitor and analyzed. User-initiated recording may obtain a current ECG reading, and is actively initiated by a user, such as by contacting the common electrodes 110, 112 with two digits of the hand opposite the wrist on which the device 100 is being worn, along with contacting another skin point with the third electrode 114. Looping monitoring is a continuous monitoring that is constantly monitoring ECG readings on a loop or the like, which triggers a recording of a specific time before and after a cardiac event of abnormality is detected. For example, when a looping device detects an abnormality, in one embodiment it triggers the device to begin recording and looks back 30 seconds and puts that ECG data along with 3-5 minutes of current data into memory.

Wearable device 100 can be provided as a Holter Monitor or as an event recorder, as a looping event recorder or a patient initiated event recorder, or may be provided as a mobile cardiac telemetry (MCT) device. Device actively sends ECG data in a non-stop transmission, usually over a cellular network. Data can be analyzed non-stop, actively. This type of device is generally worn for 30 days. When the wearable device 100 is provided as a patient initiated device, the user begins operation by, for example, touching the two common electrodes 110, 112, and contacting third electrode 114 with a skin contact point, as described above. The embodiments of the present disclosure provide the ability to provide all types of monitoring, including event recorders (looping and non-looping, user- or event-activated); Holter monitoring, and MCT monitoring.

When the wearable device 100 is provided as a looping device, the body of the wearable device 100 is coupled to a device receptacle such as matching patch 200, for example by wires 202, or in another embodiment wirelessly to wearable device 100. Matching patch 200 is attached to a chest of the user, as shown in FIG. 14A. The patch, in one embodiment provides suitable skin electrodes 204 that, in combination with the fourth electrode 116, provide suitable contacts for continuous monitoring of an ECG. The patch 200 is in one embodiment adhesive.

According to one embodiment of the present disclosure, an ECG watch as described and shown in FIG. 10 is provided. According to another embodiment of the present disclosure, an ECG recording device comprises a body (102) with bands (104) removed. In this configuration, the ECG recording device may be placed into a device receptacle such as an electro-mechanical matching patch comprising an appropriate electrode configuration which is then adhered to the chest of a user. In one embodiment, when placed into the patch, the device may be programmed to be either an event triggered device or be configured as a Holter monitor, for example a 48 hour Holter Monitor, or as an event recorder, as a looping event recorder or a patient initiated event recorder, or may be provided as a MCT device as shown further in FIG. 14B.

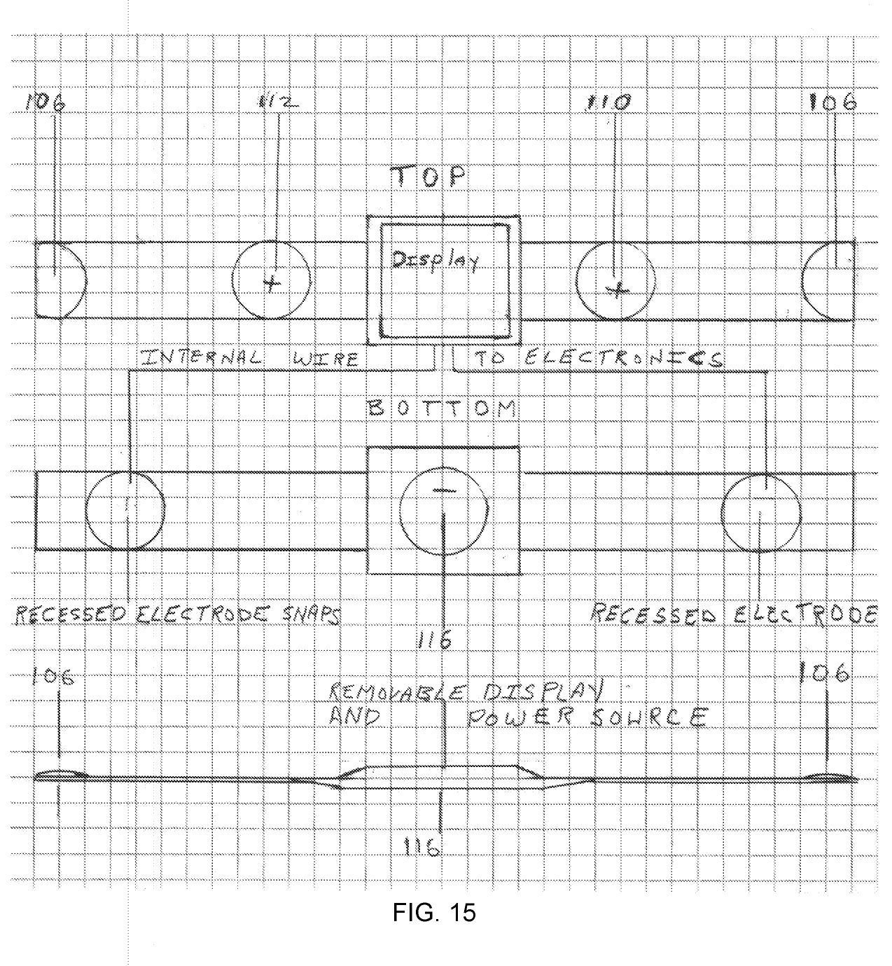

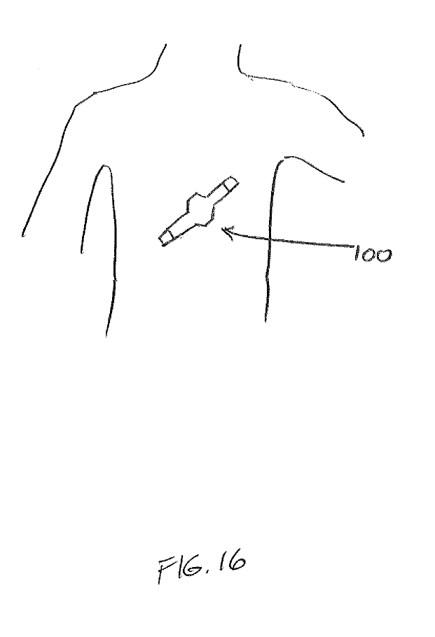

In another embodiment, as shown in FIG. 15, an ECG recording device is provided in which a wrist watch style device (100) is used in an unclasped mode. In this configuration, electrodes (106) are provided with recessed snap ECG connectors to which gel electrodes may be attached. In this configuration, the device (100) may be adhered to the chest of a user and may be used as a flat ECG recording device as is shown in FIG. 16.

In one embodiment, band 104 of the wearable device 100 is removably coupled to the body 102 of the watch using an electro-mechanical fastener.

Further features identified and described above with respect to wearable device 24 are also amenable to the embodiments of wearable device 100, and are usable therewith without departing from the scope of the disclosure. For example, LED array 36 may be incorporated into wearable device 100. Further, the LED array 36 may be colored to alert a user of a potential issue. For example, if the recorded ECG is normal, the LED array 36 can display one color, for example, green. If the recorded ECG has some anomaly or other issue, the LE array 36 can display a different color, such as yellow or red, as a warning or alert to see a doctor.

What is claimed is:

1. A wearable ECG device, comprising:
a band comprising a first and a second ECG electrode, the first and the second ECG electrodes electrically connected as a common electrode;
a device body, removably coupled to the band, having a face and a display on a first side thereof, and a third ECG electrode on an opposite face thereof; and
a clasp coupled to the band to secure the device around a wrist of a user, the clasp comprising a fourth ECG electrode, the fourth ECG electrode insulated from the band.

2. The wearable ECG device of claim 1, wherein the fourth ECG electrode is on a side of the clasp not contacting a wrist of a user when the device is worn.

3. The wearable ECG device of claim 1, wherein the first and the second ECG electrodes are on a side of the clasp not contacting a wrist of a user when the device is worn.

4. The wearable device of claim 1, wherein the third ECG electrode is on a side of the device body contacting a wrist of a user when the device is worn.

5. The wearable device of claim 1, wherein the device is configured to be used in an unclasped mode.

6. The wearable device of claim 5, wherein in the unclasped mode, the device is configured to attach to a chest of a user to provide either an event triggered ECG monitoring solution or a looping monitoring solution.

7. The wearable device of claim 1, wherein the device body is configured to be removable from the band and accommodated in a device receptacle, wherein the device receptacle comprises an electrode configuration for contact with skin of a user, the electro-mechanical matching patch configured to receive the device body therein.

8. The wearable device of claim 7, wherein the device receptacle comprises an electro-mechanical matching patch configured to receive the device body therein, and having three electrodes for contact with skin of a user, to provide a four electrode ECG when the third ECG electrode is touched by a user.

9. The wearable device of claim 1, wherein the first electrode and the second electrode are provided with recessed snap ECG connectors to which gel electrodes may be attached.

10. The wearable device of claim 6, wherein the looping monitoring solution is a Holter Monitor.

11. The wearable device of claim 1, wherein the device is configured as a mobile cardiac telemetry device.

12. An ECG device, comprising:
a device body having a face and a display on a first side thereof, and an ECG electrode on an opposite face thereof; and
an electro-mechanical matching patch comprising an electrode configuration for contact with skin of a user, the electro-mechanical matching patch configured to receive the device body therein.

13. The ECG device of claim 12, wherein the electrode configuration for the electro-mechanical patch comprises three electrodes in the electro-mechanical patch are provided on a side of the patch that contacts skin of a user.

14. The ECG device of claim 13, wherein the single electrode is provided on a side of the device body not contacting skin of a user.

15. A method of operating a wearable ECG device, comprising:
removably coupling device body having a single electrode to a device receptacle;
providing three electrodes in the device receptacle, two of the three electrodes electrically connected as common electrodes, and a third of the three electrodes insulated from the two of the three electrodes and the single electrode;
contacting the two common electrodes with two parts of a user body, and the single electrode and the third of the three electrodes with other parts of a user body.

16. The method of claim 15, wherein providing three electrodes in the device receptacle comprises providing a band removably coupleable to the device body, wherein the two of the three electrodes connected as a common electrode are provided on a first portion of a first side of the band, and wherein the third of the three electrodes is provided on a second portion of the first side of the band electrically isolated from the first portion of the first side of the band.

17. The method of claim 16, and further comprising adhering the band holding the device body to skin of a user to contact the three electrodes of the band to the skin of the user.

18. The method of claim 15, wherein the wearable ECG device is configured to provide a recording device as an event recorder, a Holter Monitor, or a mobile cardiac telemetry monitoring solution.

19. The method of claim 15, wherein providing three electrodes in the device receptacle comprises providing an electro-mechanical patch removably coupleable to the device body, wherein the three electrodes in the electro-mechanical patch are provided on a side of the patch that contacts skin of a user, and the single electrode is provided on a side of the device body not contacting skin of a user.

20. The method of claim 15, wherein the ECG device is configured to be either an event triggered device or a looping monitor.

21. The method of claim 20, wherein the looping monitor is configured as a Holter Monitor.
